(19) United States
(12) Patent Application Publication
(10) Pub. No.: US 2009/0069569 A1
(43) Pub. Date: Mar. 12, 2009

(54) CYCLOADDITION OF AZIDES AND ALKYNES

(75) Inventors: Steven P. Nolan, Tarragona (ES); Silvia Diez-Gonzalez, Barakaldo-Vizcaya (ES)

Correspondence Address: McGLINCHEY STAFFORD, PLLC;Attn: IP Group, 301 Main Street, 14th Floor, BATON ROUGE, LA, 70802, US

(73) Assignees: Institut Catala d'Investigacio Quimica, Tarragona (ES); Institucio Catalana de Recerca i Estudis Avancats, Barcelona (ES)

(21) Appl. No.: 12/209,323

(22) Filed: Sep. 12, 2008

Related U.S. Application Data
(60) Provisional application No. 60/971,779, filed on Sep. 12, 2007.

(51) Int. Cl. C07D 249/04

(52) U.S. Cl. 548/255

(57) ABSTRACT

This invention provides a process which comprises contacting, in a reaction zone, at least one organic azide, at least one alkyne, and at least one N-heterocyclic carbene copper compound in which the ligands are either (i) a halide and an N-heterocyclic carbene or (ii) two N-heterocyclic carbenes and a BF₄⁻ or PF₆⁻ anion, to form a 1,2,3-triazole in which at least the 1 and 4 positions each has a substituent. The N-heterocyclic carbene either an imidazol-2-ylidene in which the 1 and the 3 positions each has a substituent which has at least one carbon atom, or a 4,5-dihydro-imidazol-2-ylidene in which the 1 and the 3 positions each has a substituent which has at least one carbon atom.

### REFERENCE TO RELATED APPLICATION

This application claims priority from U.S. Application No. 60/971,779, filed Sep. 12, 2007, the disclosure of which is incorporated herein by reference.

### TECHNICAL FIELD

This invention relates to copper-catalyzed cycloaddition of azides and alkynes.

### BACKGROUND

Cycloaddition of azides and alkynes to yield 1,2,3-triazoles is a type of Huisgen cycloaddition. Most often, catalytic systems enabling this transformation consist of a copper(II) salt and a reducing agent. Metallic copper or copper clusters have also been employed. Copper(I) has also been reported in the catalysis of this process. One report of copper(I) catalyzed cycloaddition of azides and terminal alkynes utilized cuprous halides in reactions in which the alkyne was on a support, but the reaction did not work when both the azide and the alkyne were in solution; see Tomøe et al., J. Org. Chem., 2002, 67, 3057-3064. Tomøe et al. reported that the solution-phase reaction, using simple copper(I) salts in the presence of a nitrogen base, resulted in cross-coupling of the terminal alkynes, along with other by-products (J. Org. Chem., 2002, 67, 3057-3064). Another report of copper(I) catalysis of cycloadditions of azides and terminal alkynes employed copper(I) generated in situ from copper(II) salts, see Rostovtsev et al., Angew. Chemie Int. Ed. Engl., 2002, 41, 2596-2599.

### SUMMARY OF THE INVENTION

Pursuant to this invention, cycloaddition of organic azides and alkynes to form 1,2,3-triazoles is provided. Surprisingly, internal alkynes as well as terminal alkynes can be used to form such cycloaddition products. When a terminal alkyne is used, a 1,4-substituted 1,2,3-triazole is obtained, and when an internal alkyne is used, a 1,4,5-substituted 1,2,3-triazole is obtained. To date, other regiochemistries have not been seen using this process, and very few side products have been observed. The catalysts in the processes of this invention are copper(I) compounds in which the ligands are either (i) a halide and an N-heterocyclic carbene or (ii) two N-heterocyclic carbenes and a BF₄⁻ or PF₆⁻ anion. The processes of this invention are robust to many types of solvents, including water, and the processes can be conducted in the absence of ancillary solvent. Another advantage is that the presence of oxygen is not detrimental to the processes of this invention. The processes of this invention are considered to fall under the umbrella of “click” chemistry, reactions in which carbon-heteroatom-carbon links are made, with such reactions being stereospecific, insensitive to oxygen and water, and able to produce high yields of the product. For further details on “click” chemistry, see Kolb et al., Angew. Chemie Int. Ed. Engl., 2001, 40, 2004-2021.

An embodiment of this invention is a process which comprises contacting, in a reaction zone, at least one organic azide, at least one alkyne, and at least one N-heterocyclic carbene copper compound in which the ligands are either (i) a halide and an N-heterocyclic carbene or (ii) two N-heterocyclic carbenes and a BF₄⁻ or PF₆⁻ anion, to form a 1,2,3-triazole in which at least the 1 and 4 positions each has a substituent. The N-heterocyclic carbene is either an imidazol-2-ylidene in which the 1 and the 3 positions each has a substituent which has at least one carbon atom, or a 4,5-dihydro-imidazol-2-ylidene in which the 1 and the 3 positions each has a substituent which has at least one carbon atom.

These and other features of this invention will be still further apparent from the ensuing description and appended claims.

### FURTHER DETAILED DESCRIPTION OF THE INVENTION

Throughout this document, the term ‘reaction zone’ means any place where the organic azide, alkyne, and catalyst come together. As used throughout this document, the term ‘catalyst’ refers to the N-heterocyclic carbene copper compound, and the term ‘N-heterocyclic carbene copper compound’ means the copper(I) complexes in which the ligands are either (i) a halide and an N-heterocyclic carbene or (ii) two N-heterocyclic carbenes and a BF₄⁻ or PF₆⁻ anion. Throughout this document, the term “mol %” is used as an abbreviation for mole percent.

The reaction in the processes of this invention can be represented by the following equation:

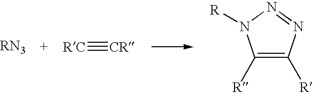

In the above equation, when R″═H, the alkyne is a terminal alkyne. The reaction shown in the equation takes place in the presence of a N-heterocyclic carbene copper compound in which the ligands are either (i) a halide and an N-heterocyclic carbene or (ii) two N-heterocyclic carbenes and a BF₄⁻ or PF₆⁻ anion. A solvent is optional for the above reaction. The R, R′, and R″ groups, as well as the N-heterocyclic carbene copper compounds, are as detailed below. In the 1,2,3-triazole formed in the reaction, when the alkyne is a terminal alkyne, the 1 and 4 positions each has a substituent; the substituent at the 1 position (R) is from the azide, and the substituent at the 4 position (R′) is from the terminal alkyne. For internal alkynes, in the 1,2,3-triazole formed in the reaction, the 1, 4 and 5 positions each has a substituent; the substituent at the 1 position (R) is from the azide, and the substituents at the 4 (R′) and 5 (R″) positions are from the internal alkyne.

The types of organic azides employed in this invention include alkyl azides, ether azides, aryl azides, and aralkyl azides. One or more functional groups, including cyano groups and nitro groups, may be present in an aryl azide, in an aralkyl azide, or in an alkyl azide. The alkyl portion of the alkyl azides can be a branched, straight chain, or cyclic group. Typically, the alkyl azides have one to about fifteen carbon atoms, and preferably about three to about ten carbon atoms. In the etheric portion of an ether azide, there can be more than one ether linkage. Ether azides generally have about three to about fifteen carbon atoms, and preferably about five to about ten carbon atoms. Preferred types of azides include aryl azides and aralkyl azides. Mixtures of any two or more organic azides can be used, if desired. The use of mixtures of organic azides will yield a mixture of triazoles.

Suitable alkyl azides include, but are not limited to, methyl azide, ethyl azide, n-propyl azide, isopropyl azide, cyclopropyl azide, 3-cyanopropyl azide, n-butyl azide, sec-butyl azide, tert-butyl azide, cyclobutyl azide, 4-cyanobutyl azide, pentyl azide, 3-cyanopentyl azide, cyclopentyl azide, 2,2-dimethylpropyl azide, hexyl azide, cyclohexyl azide, 4-cyanocyclohexyl azide, methylcycxlohexyl azide, heptyl azide, octyl azide, cyclooctyl azide, nonyl azide, and decyl azide. Preferred alkyl azides include methyl azide, 3-cyanopropyl azide, and heptyl azide. It is noted that methyl azide, ethyl azide, and n-propyl azide are quite explosive, and thus care should be exercised in their handling.

Examples of ether azides that can be used in the practice of this invention include 3,3-dimethoxypropyl azide, 3,3-diethoxypropyl azide, 4-butyloxybutyl azide, 4-propoxypentyl azide, 5-methoxyhexyl azide, 4-(2-tetrahydrofuranyl)-butyl azide, 2-[2-(1,3-dioxolanyl)]-ethyl azide, 2-[2-(1,3-dioxanyl)]-ethyl azide, 3-[2-(1,3-dioxolanyl)]-butyl azide, 4-[2-(1,3-dioxanyl)]-pentyl azide, 6-(2-tetrahydrofuranyl)-hexyl azide, and the like. Preferred ether azides include 3,3-diethoxypropyl azide and 2-[2-(1,3-dioxolanyl)]-ethyl azide.

Aryl azides that can be used in this invention include, but are not limited to, phenyl azide, 2-cyanophenyl azide, 4-cyanophenyl azide, 3-nitrophenyl azide, 4-nitrophenyl azide, tolyl azide, 2-methyl-4-nitrophenyl azide, 3-methyl-5-cyanophenyl azide, 2,5-dimethylphenyl azide, biphenyl azide, 3-nitro-biphenyl azide, 4′-cyanobiphenyl azide, naphthyl azide, 1-(4-cyano)naphthyl azide, 2-(6-nitro)naphthyl azide, 1-anthryl azide, 1-(10-cyano)anthryl azide, 2-(6-nitro)anthryl azide, 2-phenanthryl azide, 1-(6-cyano)phenanthryl azide, and 2-(9-nitro)-phenanthryl azide. Preferred aryl azides include phenyl azide, 4-cyanophenyl azide, and 4-nitrophenyl azide.

Suitable aralkyl azides include benzyl azide, 4-methylbenzyl azide, 2-phenylethyl azide, 2-(3-cyanophenyl)ethyl azide, 2-(4-nitrophenyl)ethyl azide, 2-(2-methylphenyl)ethyl azide, 3-phenylbutyl azide, diphenylmethyl azide, 4-cyanobenzyl azide, 4-nitrobenzyl azide, 1-naphthylmethyl azide, [1-(6-cyano)-naphthyl]ethyl azide, 2-naphthylethyl azide, [2-(4-nitro)-naphthyl]methyl azide, and the like. Preferred aralkyl azides include benzyl azide, 2-phenylethyl azide, 4-cyanobenzyl azide, and 4-nitrobenzyl azide.

Many of the organic azides that can be used in this invention are relatively stable, and thus can be purchased or prepared ahead of time and stored until needed. Some of the organic azides that can be used in the practice of this invention are not stable in the sense that they cannot be stored for later use. In general, the organic azides which are not stable in this sense are those of low molecular weight (i.e., those with fewer than about four carbon atoms); see in this connection Scriven and Tumbull, Chem. Rev., 1988, 88, 297-368. Such organic azides can be generated shortly before, or preferably during, the processes of this invention from an organic halide and an alkali metal azide.

For the organic halide, the organic group of the organic halide can be alkyl, ether, aryl, or aralkyl; characteristics and preferences of these organic groups are as described above for the organic azides. The organic halide can be a chloride, bromide, or iodide. Organic bromides and organic iodides are preferred.

The alkali metal azide can be lithium azide, sodium azide, or potassium azide; sodium azide is preferred. Normally, approximately equimolar amounts of the organic halide and the alkali metal azide are used; a slight excess of the alkali metal azide (e.g., about 1.01 to about 1.10 moles of alkali metal azide per mole of organic halide) is preferred.

Both terminal alkynes and internal alkynes can be used in the practice of this invention; both types of alkyne can contain functional groups. Suitable functional groups in these alkynes include carbon-carbon double bonds, ether groups, ester groups, ketyl groups, hydroxyl groups, chlorine atoms, fluorine atoms, trihydrocarbylsilyl groups, nitrogen atoms (e.g., as amino groups), and the like. In the practice of this invention, terminal alkynes typically have three to about twenty carbon atoms, and preferably about five to about twelve carbon atoms. When expressed as RC≡CH, groups R that may be part of a terminal alkyne in this invention include alkyl groups (straight chain, cyclic, or, preferably, branched), alkenyl groups (straight chain, branched, or, preferably, cyclic), aryl groups, and silyl groups. The internal alkynes in the practice of this invention typically have four to about twenty carbon atoms, and preferably about six to about twelve carbon atoms. For the internal alkynes used in this invention, when expressed as R¹C≡CR², groups R¹ and R², which may be the same or different, include alkyl groups (straight chain, branched, or cyclic), alkenyl groups (straight chain, branched, or cyclic), aryl groups, and silyl groups. Mixtures of any two or more alkynes can be used, if desired. The use of mixtures of alkynes will yield a mixture of triazoles.

Examples of terminal alkynes that can be used in the practice of this invention include, but are not limited to, 1-propyne, cyclopropylacetylene, 1-butyne, 1-pentyne, 3,3-dimethyl-1-butyne, 1-hexyne, cyclohexylacetylene, 1-heptyne, 3-cyclopentyl-1-propyne, 1-octyne, 1-nonyne, 1-decyne, 2-methyl-1-buten-3-yne, 3-penten-1-yne, 3-hexen-1-yne, 2-ethynylcyclopentene, 1-ethynylcyclohexene, 3-ethyl-3-penten-1-yne, 5-decen-1-yne, phenylacetylene, 3-tert-butylphenylacetylene, 1-ethyl-4-ethynylbenzene, 4-phenyl-1-butyne, 4-methoxyphenylacetylene, 1-ethynyl-3,5-dimethoxybenzene, 1-ethynyl-4-phenoxybenzene, 3-chloropropyne (propargyl chloride), 4-chlorobutyne, 3-chloro-3-methyl-1-butyne, 5-chloropentyne, 4-chlorohexyne, 6-chlorohexyne, 7-chloro-3-heptyne, 2-fluorophenylacetylene, 3-fluorophenylacetylene, 4-fluorophenylacetylene, 2-methyl-3-fluorophenylacetylene, 4-ethynylbiphenyl, 1-ethynylnaphthalene, 2-ethynylnaphthalene, 2-ethynyl-6-methoxynaphthalene, 1-ethynylanthracene, 2-ethynyl-6-methoxyanthracene, 9-ethynylphenanthrene, 2-ethynyl-6-fluorophenanthrene, 2-propyn-1-ol (propargyl alcohol), 3-butyn-1-ol, 2-methyl-3-butyn-2-ol, 1-pentyn-4-ol, 1-hexyn-3-ol, 1-hexyn-5-ol, 1-ethynyl-1-cyclohexanol, 1-octyn-3-ol, hydroxyphenylacetylene, 3-hydroxy-3-phenyl-1-propyne, 2-phenyl-3-butyn-2-ol, 3-methoxypropyne, 3-propoxypropyne, 3-tert-butoxy-1-butyne, methyl propiolate, ethyl propiolate, 3-butyn-2-one, 1-pentyn-3-one, 4-methyl-1-pentyn-3-one, 2-pentyn-4-one, 1-hexyn-3-one, 3-hexyn-2-one, 2-hexyn-4-one, 3-heptyn-2-one, (trimethylsilyl)acetylene, (triethylsilyl)acetylene, (triisopropylsilyl)acetylene, (dimethylphenylsilyl)acetylene, (methyldiphenylsilyl)acetylene, (triphenylsilyl)acetylene, 3-(dimethylamino)propyne, 3-(dipropylamino)propyne, 4-(diethylamino)-2-butyne, 5-(dimethylamino)-3-pentyne, 5-(diethylamino)-3-pentyne, 2-ethynylpyridine, 3-ethynylpyridine, and 4-ethynylpyridine. Preferred terminal alkynes include 3,3-dimethyl-1-butyne, 1-ethynylcyclohexene, phenylacetylene, 5-chloropentyne, 2-methyl-3-butyn-2-ol, 2-propyn-1-ol, 4-methoxyphenylacetylene, ethyl propiolate, (trimethylsilyl)acetylene, 3-(dimethylamino)propyne, 2-ethynylpyridine, and 3-ethynylpyridine.

Suitable internal alkynes in the practice of this invention include 2-butyne, 4-methyl-2-pentyne, 2-hexyne, 3-hexyne, 4-octyne, 5-decyne, diphenylacetylene, dinaphthylacetylene, 1-phenyl-1-propyne, 1-phenyl-1-pentyne, 1-phenyl-1-hexyne, 2-methyl-1-penten-3-yne, 4-hexen-2-yne, 4-ethyl-4-hexen-2-yne, 3-decen-5-yne, 1-cyclopentenyl-1-butyne, 1-cyclohexenyl-1-propyne, 1-methoxy-3-pentyne, 2-propoxy-3-pentyne, 1-ethoxy-3-hexyne, 3-isopropoxy-4-heptyne, 2-tert-butoxy-4-octyne, di(methoxyphenyl)acetylene, di(3,5-dimethoxyphenyl)acetylene, 3-pentyn-1-ol, hex-4-yn-1-ol, 2-methyl-3-hexyn-2-ol, 4-heptyn-2-ol, 3-octyn-2-ol, 3-nonyn-1-ol, 6-nonyn-1-ol, 3-decyn-1-ol, 1-phenyl-1-hexyn-3-ol, ethyl-2-butynoate, methyl-pent-2-yn-1-oate, ethyl hex-2-ynoate, ethyl hep-2-ynoate, pentyl octyn-2-oate, 1-trimethylsilyl-1-propyne, 1-triisopropylsilyl-1-propyne, 1-triethylsilyl-1-pentyne, 1-(trimethylsilyl)-2-phenylacetylene, and the like. Preferred internal alkynes include 3-hexyne and diphenylacetylene.

In the N-heterocyclic carbene copper halide, the halide is chloride, bromide, or iodide; preferably, the halide is chloride or bromide, and more preferably the halide is bromide. The copper is copper(I); that is, copper formally in the +1 oxidation state. Mixtures of two or more catalysts can be used.

In the N-heterocyclic carbene copper compounds in which there are two N-heterocyclic carbene ligands and a BF₄⁻ or PF₆⁻ anion, there is no preference for either the BF₄⁻ anion or PF₆⁻ anion. The copper is copper(I); that is, copper formally in the +1 oxidation state. Mixtures of two or more catalysts can be used.

The N-heterocyclic carbene can be unsaturated (imidazol-2-ylidene) or saturated (4,5-dihydro-imidazol-2-ylidene). Saturated carbenes are preferred. Substituents at the 1 and 3 positions, which may be the same or different, generally have one to about twenty carbon atoms; preferably such substituents have three to about fifteen carbon atoms. At least one, and preferably both, of the substituents on the N-heterocyclic carbene is each, independently, an aryl group or an alkyl group having at least 3 carbon atoms. Alkyl group substituents are preferably secondary or tertiary groups. Aryl group substituents are preferably substituted by an alkyl group in each ortho position; a preferred aryl moiety is a phenyl group.

Suitable substituent groups for the 1 and 3 positions of the carbene include, but are not limited to, methyl, ethyl, n-propyl, isopropyl, cyclopropyl, n-butyl, sec-butyl, tert-butyl, cyclobutyl, n-pentyl, 3-pentyl, 2,2-dimethylpropyl (neopentyl), cyclopentyl, cyclohexyl, methylcyclohexyl, 2,5-dimethylhex-2-yl, cyclooctyl, norbornyl, adamantyl, benzyl, phenyl, biphenylyl, naphthyl, anthracenyl, tolyl, 3,5-dimethylphenyl, 2,4,6-trimethylphenyl (mesityl), 2,6-di(isopropyl)phenyl, 2,4,6-tri(isopropyl)phenyl, 2,4,6-tri(isopropyl)phenylmethyl, 2,6-di(tert-butyl)phenyl, triphenylmethyl, and 1,3-dimethyl-2-naphthyl groups. Preferred carbene substituent groups include adamantyl, 2,4,6-trimethylphenyl, and 2,6-di(isopropyl)phenyl groups.

Examples of suitable N-heterocyclic carbenes include N,N′-dimethyl-imidazol-2-ylidene, N,N′-diethyl-4,5-dihydro-imidazol-2-ylidene, N,N′-di-n-propyl-imidazol-2-ylidene, N,N′-di(isopropyl)-4,5-dihydro-imidazol-2-ylidene, N,N′-di-tert-butyl-imidazol-2-ylidene, N,N′-di(2,2-dimethylpropyl)-4,5-dihydro-imidazol-2-ylidene, N,N′-dicyclopentyl-imidazol-2-ylidene, N,N′-di(cyclohexyl)-imidazol-2-ylidene, N,N′-di(cyclohexyl)-4,5-dihydro-imidazol-2-ylidene, N,N′-di(methylcyclohexyl)-4,5-dihydro-imidazol-2-ylidene, N,N′-di(adamantyl)-imidazol-2-ylidene, N,N′-dibenzyl-4,5-dihydro-imidazol-2-ylidene, N,N′-dinaphthyl-imidazol-2-ylidene, N,N′-ditolyl-4,5-dihydro-imidazol-2-ylidene, N,N′-bis{2,6-di(isopropyl)phenyl}-imidazol-2-ylidene, N,N′-bis[2,6-di(isopropyl)phenyl]-4,5-dihydro-imidazol-2-ylidene, N,N′-bis(2,4,6-trimethylphenyl)-imidazol-2-ylidene, N,N′-bis(2,4,6-trimethylphenyl)-4,5-dihydro-imidazol-2-ylidene, N,N′-bis[2,4,6-tri(isopropyl)phenyl]-imidazol-2-ylidene, N,N′-bis[2,6-di(tert-butyl)phenyl]-4,5-dihydro-imidazol-2-ylidene, N,N′-bis(triphenylmethyl)-imidazol-2-ylidene, N,N′-bis(1,3-dimethyl-2-naphthyl)-4,5-dihydro-imidazol-2-ylidene, and the like. Preferred N-heterocyclic carbenes include N,N′-di(cyclohexyl)-imidazol-2-ylidene, N,N′-di(adamantyl)-imidazol-2-ylidene, N,N′-bis[2,6-di(isopropyl)phenyl]-imidazol-2-ylidene, N,N′-bis(2,4,6-trimethylphenyl)-imidazol-2-ylidene, N,N′-bis[2,6-di(isopropyl)phenyl]-4,5-dihydro-imidazol-2-ylidene, and N,N′-bis(2,4,6-trimethylphenyl)-4,5-dihydro-imidazol-2-ylidene.

N-heterocyclic carbene copper halides that can be used in the practice of this invention include, but are not limited to, [N,N′-dimethyl-imidazol-2-ylidene]copper chloride, [N,N′-diethyl-imidazol-2-ylidene]copper bromide, [N,N′-di-n-propyl-4,5-dihydro-imidazol-2-ylidene]copper chloride, [N,N′-di(isopropyl)-4,5-dihydro-imidazol-2-ylidene]copper bromide, [N,N′-di-sec-butyl-4,5-dihydro-imidazol-2-ylidene]copper iodide, [N,N′-di-tert-butyl-imidazol-2-ylidene]copper chloride, [N,N′-di-3-pentyl-imidazol-2-ylidene]copper iodide, [N,N′-di(2,2-dimethylpropyl)-imidazol-2-ylidene]copper bromide, [N,N′-dicyclopentyl-4,5-dihydro-imidazol-2-ylidene]copper chloride, [N,N′-di(cyclohexyl)-imidazol-2-ylidene]copper iodide, [N,N′-di(methylcyclohexyl)-4,5-dihydro-imidazol-2-ylidene]copper bromide, [N,N′-di(adamantyl)-imidazol-2-ylidene]copper chloride, [N,N′-dibenzyl-imidazol-2-ylidene]copper bromide, [N,N′-diphenyl-imidazol-2-ylidene]copper iodide, [N,N′-dinaphthyl-4,5-dihydro-imidazol-2-ylidene]copper chloride, [N,N′-dianthracenyl-imidazol-2-ylidene]copper iodide, [N,N′-ditolyl-4,5-dihydro-imidazol-2-ylidene]copper bromide, [N,N′-bis(biphenylyl)-imidazol-2-ylidene]copper iodide, [N,N′-bis{2,4,6-tri(isopropyl)phenyl}-imidazol-2-ylidene]copper chloride, [N,N′-bis{2,6-di(tert-butyl)phenyl}-imidazol-2-ylidene]copper bromide, [N,N′-bis(triphenylmethyl)-4,5-dihydro-imidazol-2-ylidene]copper chloride, [N,N′-bis(1,3-dimethyl-2-naphthyl)-4,5-dihydro-imidazol-2-ylidene]copper bromide, [N,N′-bis{2,6-di(isopropyl)phenyl}-imidazol-2-ylidene]copper chloride, [N,N′-bis{2,6-di(isopropyl)phenyl}-imidazol-2-ylidene]copper bromide, [N,N′-bis{2,6-di(isopropyl)phenyl}-imidazol-2-ylidene]copper iodide, [N,N′-bis{2,6-di(isopropyl)phenyl}-4,5-dihydro-imidazol-2-ylidene]copper chloride, [N,N′-bis{2,6-di(isopropyl)phenyl}-4,5-dihydro-imidazol-2-ylidene]copper bromide, [N,N′-bis{2,6-di(isopropyl)phenyl}-4,5-dihydro-imidazol-2-ylidene]copper iodide, [N,N′-bis(2,4,6-trimethylphenyl)-imidazol-2-ylidene]copper chloride, [N,N′-bis(2,4,6-trimethylphenyl)-imidazol-2-ylidene]copper bromide, [N,N′-bis(2,4,6-trimethylphenyl)-imidazol-2-ylidene]copper iodide, [N,N′-bis(2,4,6-trimethylphenyl)-4,5-dihydro-imidazol-2-ylidene]copper chloride, [N,N′-bis(2,4,6-trimethylphenyl)-4,5-dihydro-imidazol-2-ylidene]copper bromide, and [N,N′-bis(2,4,6-trimethylphenyl)-4,5-dihydro-imidazol-2-ylidene]copper iodide.

N-heterocyclic carbene copper halides that are preferred in the practice of this invention include [N,N′-bis{2,6-di(isopropyl)phenyl}-imidazol-2-ylidene]copper chloride, [N,N′-bis{2,6-di(isopropyl)phenyl}-imidazol-2-ylidene]copper bromide, [N,N′-bis(2,4,6-trimethylphenyl)-imidazol-2-ylidene]copper chloride, [N,N′-bis(2,4,6-trimethylphenyl)-imidazol-2-ylidene]copper bromide, [N,N′-bis(2,4,6-trimethylphenyl)-4,5-dihydro-imidazol-2-ylidene]copper chloride, and [N,N′-bis(2,4,6-trimethylphenyl)-4,5-dihydro-imidazol-2-ylidene]copper bromide.

N-heterocyclic carbene copper compounds in which there are two N-heterocyclic carbene ligands and a BF₄⁻ or PF₆⁻ anion that can be used in the practice of this invention include, but are not limited to, bis(N,N′-di-tert-butyl-imidazol-2-ylidene)copper hexafluorophosphate, bis(N,N′-di-tert-butyl-imidazol-2-ylidene)copper tetrafluoroborate, bis(N,N′-di(cyclohexyl)-imidazol-2-ylidene)copper hexafluorophosphate, bis(N,N′-di(cyclohexyl)-imidazol-2-ylidene)copper tetrafluoroborate, bis(N,N′-di(adamantyl)-imidazol-2-ylidene)copper hexafluorophosphate, bis(N,N′-di(adamantyl)-imidazol-2-ylidene)copper tetrafluoroborate, bis(N,N′-bis[2,6-di(isopropyl)phenyl]-imidazol-2-ylidene)copper hexafluorophosphate, bis(N,N′-bis[2,6-di(isopropyl)phenyl]-imidazol-2-ylidene)copper tetrafluoroborate, bis(N,N′-bis(2,4,6-trimethylphenyl)-imidazol-2-ylidene)copper hexafluorophosphate, bis(N,N′-bis(2,4,6-trimethylphenyl)-imidazol-2-ylidene)copper tetrafluoroborate, bis(N,N′-bis[2,6-di(isopropyl)phenyl]-4,5-dihydro-imidazol-2-ylidene)copper hexafluorophosphate, bis(N,N′-di-tert-butyl-4,5-dihydro-imidazol-2-ylidene)copper hexafluorophosphate, bis(N,N′-di-tert-butyl-4,5-dihydro-imidazol-2-ylidene)copper tetrafluoroborate, bis(N,N′-di(cyclohexyl)-4,5-dihydro-imidazol-2-ylidene)copper hexafluorophosphate, bis(N,N′-di(cyclohexyl)-4,5-dihydro-imidazol-2-ylidene)copper tetrafluoroborate, bis(N,N′-di(adamantyl)-4,5-dihydro-imidazol-2-ylidene)copper hexafluorophosphate, bis(N,N′-di(adamantyl)-4,5-dihydro-imidazol-2-ylidene)copper tetrafluoroborate, bis(N,N′-bis[2,6-di(isopropyl)phenyl]-4,5-dihydro-imidazol-2-ylidene)copper hexafluorophosphate, bis(N,N′-bis[2,6-di(isopropyl)phenyl]-4,5-dihydro-imidazol-2-ylidene)copper tetrafluoroborate, bis(N,N′-bis(2,4,6-trimethylphenyl)-4,5-dihydro-imidazol-2-ylidene)copper hexafluorophosphate, and bis(N,N′-bis(2,4,6-trimethylphenyl)-4,5-dihydro-imidazol-2-ylidene)copper tetrafluoroborate.

N-heterocyclic carbene copper compounds in which there are two N-heterocyclic carbene ligands and a BF₄⁻ or PF₆⁻ anion that are preferred in the practice of this invention include bis(N,N′-di(cyclohexyl)-imidazol-2-ylidene)copper hexafluorophosphate, bis(N,N′-di(cyclohexyl)-imidazol-2-ylidene)copper tetrafluoroborate, bis(N,N′-di(adamantyl)-imidazol-2-ylidene) copper hexafluorophosphate, bis(N,N′-di(adamantyl)-imidazol-2-ylidene) copper tetrafluoroborate, bis(N,N′-bis[2,6-di(isopropyl)phenyl]-imidazol-2-ylidene)copper hexafluorophosphate, bis(N,N′-bis[2,6-di(isopropyl)phenyl]-imidazol-2-ylidene)copper tetrafluoroborate, bis(N,N′-bis(2,4,6-trimethylphenyl)-imidazol-2-ylidene)copper hexafluorophosphate, bis(N,N′-bis(2,4,6-trimethylphenyl)-imidazol-2-ylidene)copper tetrafluoroborate, bis(N,N′-bis[2,6-di(isopropyl)phenyl]-4,5-dihydro-imidazol-2-ylidene)copper hexafluorophosphate, bis(N,N′-bis[2,6-di(isopropyl)phenyl]-4,5-dihydro-imidazol-2-ylidene)copper tetrafluoroborate, bis(N,N′-bis(2,4,6-trimethylphenyl)-4,5-dihydro-imidazol-2-ylidene)copper hexafluorophosphate, and bis(N,N′-bis(2,4,6-trimethylphenyl)-4,5-dihydro-imidazol-2-ylidene)copper tetrafluoroborate.

Solvent is not usually necessary in the processes of this invention. Generally, it is recommended and preferred to conduct the processes of this invention either in water or without solvent. While solvents other than water can be used, adverse effects on product yield were observed in tetrahydrofuran, dichloromethane, and tert-butanol, when the catalyst was an N-heterocyclic carbene copper halide. For terminal alkynes, when the catalyst was an N-heterocyclic carbene copper halide, better results (higher yields and shorter reaction times) have been obtained in water than in mixtures of water and organic solvents. When the catalyst was a N-heterocyclic carbene copper compound in which there were two N-heterocyclic carbene ligands and a BF₄⁻ or PF₆⁻ anion, complete conversions were obtained in two hours or less in water, dimethylsulfoxide (DMSO), dimethylformamide (DMF), tetrahydrofuran (THF), acetone, and acetonitrile. In contrast, reactions were slow in alcohols for catalysts in which there were two N-heterocyclic carbene ligands and a BF₄⁻ or PF₆⁻ anion.

When a solvent is employed, concentrations of the organic azide and the alkyne are each, independently, typically about 0.5 molar or higher, preferably about 1 molar or higher. Lower concentrations can be employed, but no particular advantage is expected in more dilute solution.

In the processes of this invention, one mole of organic azide and one mole of alkyne are consumed for each mole of 1,2,3-triazole produced. Thus it is recommended and preferred to use approximately equimolar amounts of organic azide and alkyne. The amounts of organic azide and of alkyne are preferably such that the alkyne is in slight excess relative to the organic azide. More preferably, the alkyne is in the range of about 1.01 to about 1.10 moles per mole of organic azide.

Catalytic amounts of the N-heterocyclic carbene copper compound are used. More particularly, the amount of N-heterocyclic carbene copper compound is usually in the range of about 0.2 mol % to about 10 mol % relative to the organic azide. Preferably, about 0.5 mol % to about 5 mol % of N-heterocyclic carbene copper compound relative to the organic azide is employed. When the alkyne is a terminal alkyne, more preferred amounts of the N-heterocyclic carbene copper compound are in the range of about 0.5 mol % to about 3 mol % relative to the organic azide. When the alkyne is an internal alkyne, more preferred amounts of the N-heterocyclic carbene copper compound are in the range of about 1 mol % to about 5 mol % relative to the organic azide.

Temperatures during the processes of the invention generally range from about room temperature (˜20° C.) to about 95° C., and preferably from about room temperature to about 80° C. For internal alkynes, temperatures in the range from about 50° C. to about 80° C. are more preferred. For terminal alkynes, temperatures in the range from about room temperature to about 50° C. are more preferred. To date, most of the reactions involving terminal alkynes have been successful at room temperature; warmer conditions are normally employed to shorten the reaction time.

When a reaction of an organic azide and an alkyne was carried out at 40° to 50° C. and an 8-hour reaction time at low catalyst loading (about 50 to about 300 ppm), at least for N-heterocyclic carbene copper compounds in which there are two N-heterocyclic carbene ligands and a BF₄⁻ or PF₆⁻ anion, minor amounts of the 1,5-regioisomer of the triazole were observed by ¹H NMR and by gas chromatography (GC). In contrast, at room temperature, a low catalyst loading (about 50 to about 300 ppm) yielded conversions of about 45% to about 91% to the desired 1,4- and 1,4,5-isomers, more typically of about 70% to about 90%, as observed by GC.

The organic azide, alkyne, catalyst, and optionally, solvent, can be brought together in any order, including the co-feeding of two or more of these components. Typically, the reaction zone is a vessel to which the components are introduced, although e.g., a pipe or mixer can also be the reaction zone. For organic azides which cannot be stored for later use (i.e., small organic azides), such organic azide can be formed in a place in which the alkyne and the catalyst are already present, or to which the alkyne and the catalyst are being or will be fed, or the organic azide can be formed in a separate vessel and then fed to the alkyne and the catalyst. Variations on these schemes, such as co-feeding of the organic azide, are within the scope of this invention. When the organic azide is prepared in a place in which the alkyne and the catalyst are already present, it is sometimes said to have been made in situ. As stated above, the exclusion of oxygen is not necessary during the processes of this invention.

On the laboratory scale, the reaction time for the processes of the invention can be quite short, on the order of minutes (e.g., 10 to 15 minutes) to about eighteen hours when a terminal alkyne is used. Internal alkynes react more slowly than do terminal alkynes, and thus reaction times are longer, e.g., about 48 hours on the laboratory scale. Generally, raising the reaction temperature and/or increasing the amount of catalyst often can shorten the reaction time. As noted above, in processes in which solvent is present, water may be used to shorten the reaction time for reactions involving terminal alkynes. Processes that produced 1,2,3-triazoles which were oils or which had low melting points tended to require longer reaction times. Product triazoles containing long alkyl chains are more likely to be oils. A small-scale synthesis of the desired product can verify whether the product is an oil, or provide enough product for a melting point determination before scale-up of the process for a particular set of reagents.

Generally, the 1,2,3-triazole products are solids, and can be isolated by standard methods such as precipitation or centrifugation and decantation. For 1,2,3-triazoles which are oils, isolation is typically via solvent extraction and/or chromatographic methods.

The following examples are presented for purposes of illustration, and are not intended to impose limitations on the scope of this invention.

### EXAMPLES

Abbreviations. Abbreviations used in the Examples include:
I for N-heterocyclic carbene ligands in which the N-heterocyclic ring is unsaturated (an imidazol-2-ylidene);
SI for N-heterocyclic carbene ligands in which the N-heterocyclic ring is saturated (4,5-dihydro-imidazol-2-ylidene)
Mes for mesityl groups, which are 2,4,6-trimethylphenyl groups; and
Pr for 2,6-di(isopropyl)phenyl groups;
Ad for adamantyl groups;
Cx for cyclohexyl groups; and
tBu for tert-butyl groups.
   For example, SIMes is N,N′-bis(2,4,6-trimethylphenyl)-4,5-dihydro-imidazol-2-ylidene. In this connection, when only one group is listed in such an abbreviation, it signifies that the same group is present on both nitrogen atoms of the carbene ring, unless otherwise stated.

General conditions. All reagents were used as purchased. Copper(I) bromide and sodium tert-butoxide were stored under argon in a glovebox. 1,3-Bis-(2,4,6-trimethylphenyl)imidazolium chloride (SIMes.HCl) was synthesized according to literature procedures (see A. J. Arduengo III et al., Tetrahedron 1999, 55, 14523-14534) or purchased from Strem. Flash column chromatography was performed on silica gel 60 (230-400 mesh). ¹H and ¹³C nuclear magnetic resonance (NMR) spectra were recorded on a 300 MHz spectrometer at room temperature. Chemical shifts (δ) are reported with respect to tetramethylsilane as internal standard in ppm. Assignments of some ¹H and ¹³C NMR signals rely on COSY and/or HMBC experiments. Elemental analyses were performed at Robertson Microlit Laboratories, Inc., Madison, N.J., USA.

Synthesis of [(SIMes)CuBr]. In an oven-dried vial, copper(I) bromide (0.522 g, 3.63 mmol), SIMes.HCl (0.86 g, 2.52 mmol) and sodium tert-butoxide (0.243 g, 2.52 mmol) were loaded inside a glovebox and stirred in dry THF (18 mL) overnight at room temperature outside of the glovebox. After filtration of the reaction mixture through a plug of Celite, the filtrate was mixed with hexane to form a precipitate. A second filtration afforded 0.808 g (71% yield) of the title complex as an off-white solid.

Spectroscopic and analytical data for [(SIMes)CuBr]: ¹H NMR (300 MHz, [D₆]acetone): δ=7.01 (s, 4H, H^{Ar}), 4.16 (s, 4H, NCH₂), 2.37 (s, 12H, ArCH₃), 2.29 (s, 6H, ArCH₃); ¹³C NMR (75 MHz, CDCl₃): δ=202.6 (C, NCN), 138.5 (C, C^{Ar}), 135.3 (CH, C^{Ar}), 135.0 (C, C^{Ar}), 129.7 (CH, C^{Ar}), 51.0 (CH₂, NCH₂), 21.0 (CH₃, ArCH₃), 18.0 (CH₃, ArCH₃); Elemental analysis calcd for C₂₁H₂₆BrCuN₂ (449.89): C, 56.06; H, 5.83; N, 6.23. Found: C, 55.98; H, 5.64; N, 6.21%.

Synthesis of [(SIMes)CuCl]. This synthesis is as reported in the literature; see S. Diez-González et al., J. Org. Chem. 2005, 70, 4784-4796. In a 250 mL Schlenk flask were added copper(I) chloride (1.0 g, 10.10 mmol), 1,3-bis(2,4,6-trimethylphenyl)-4,5-dihydro-imidazol-2-ylidenium chloride (SIMes-HCl, 10.10 mmol), and sodium tert-butoxide (0.97 g, 10.10 mmol). To this flask, dry tetrahydrofuran (100 mL) was added under an inert atmosphere of argon, and the mixture was magnetically stirred for 20 hours at room temperature. After the mixture was filtered through a plug of Celite and then evaporating the solvent under vacuum, a white solid was obtained. ¹H NMR (400 MHz, CDCl₃) δ=6.96 (s, 4H), 3.96 (s, 4H), 2.32 (s, 12H), 2.30 (s, 6H); ¹³C NMR (100 MHz, CDCl₃) δ=202.8, 138.7, 135.3, 135.0, 129.7, 50.9, 21.0, 18.0. Elemental analysis calcd for C₂₁H₂₆CuClN₂: C, 62.21; H, 6.46; N, 6.91. Found: C, 62.60; H, 6.52; N, 6.80%.

Synthesis of [(IMes)CuCl]. This synthesis is as reported in the literature; see S. Okamoto et al., J. Organomet. Chem. 2005, 690, 6001-6007. Tetrahydrofuran (7 mL) was added to a mixture of 1,3-bis(2,4,6-trimethylphenyl)imidazolium chloride (IMes-HCl, 1 mmol), CuCl (0.9 mmol), and sodium tert-butoxide (1 mmol). The suspension was stirred for 6 hours at room temperature, and then filtered through a pad of Celite. The filtrate was dried under vacuum. ¹H NMR (500 MHz, CDCl₃) δ=7.06 (s, 2H), 7.00 (s, 4H), 2.34 (s, 6H), 2.30 (d, 12H); ¹³C NMR (125 MHz, CDCl₃) δ=178.7, 139.2, 134.9, 134.4, 129.3, 122.2, 21.1, 17.6; IR (KBr) 2914, 1485, 1400, 1234, 1076, 932, 862, 702 cm⁻¹; Elemental analysis calcd for C₂₁H₂₄CuClN₂: C, 62.52; H, 6.00; N, 6.94. Found: C, 62.33; H, 6.16; N, 6.86%.

Synthesis of [(IPr)CuCl]. This synthesis is as reported in the literature; see H. Kaur et al., Organometallics 2004, 23, 1157-1160. In a 250 mL Schlenk flask were added copper(I) chloride (1.0 g, 10.10 mmol), 1,3-bis(2,6-diisopropylphenyl)imidazolium chloride (IPr.HCl; 4.29 g, 10.10 mmol), and sodium tert-butoxide (0.97 g, 10.10 mmol). To this flask, dry tetrahydrofuran (100 mL) was added under an inert atmosphere of argon, and the mixture was magnetically stirred for 20 hours at room temperature. After the mixture was filtered through a plug of Celite and then evaporating the solvent under vacuum, a white solid was obtained (4.59 g, 9.40 mmol, 94%). ¹H NMR: (400 MHz, acetone-d₆, ppm) δ=1.21 (d, J=6.8 Hz, 12H); 1.30 (d, J=6.8 Hz, 12H); 2.57 (hep, J=6.8 Hz, 4H); 7.12 (s, 2H). 7.29 (d, J=7.8 Hz, 4H); 7.49 (t, J=7.8 Hz, 2H). ¹³C NMR: (100 MHz, acetone-d₆, ppm) δ=182.32; 145.61; 134.41; 130.62; 124.25; 123.13; 28.76; 24.82; 23.87. Elemental analysis calcd for C₂₇H₃₆ClCuN₂: C 66.64%, H, 7.46%, N, 5.76%; found C, 66.70%, H, 7.48%, N, 6.06%.

### Example 1

Several runs were performed using benzyl azide and phenylacetylene, using different copper halide catalysts and/or solvents. In a vial fitted with a screw cap, benzyl azide (1.0 mmol), phenylacetylene (1.05 mmol) and the catalyst were loaded. The reaction was allowed to proceed at room temperature and monitored by ¹H NMR analysis of aliquots. After total consumption of benzyl azide, the solid product was collected by filtration and washed with water and pentane. It was observed that [(IMes)CuCl] gave better results than did [(IPr)CuCl], and that [(SIMes)CuCl] gave better results than [(IMes)CuCl]. [(SIMes)CuBr] gave better results than did [(SIMes)CuCl]. Results are summarized in Table 1.

TABLE 1

| | | Amount of | | | |
|---|---|---|---|---|---|
| Run | Catalyst | catalyst^{a} | Solvent (mL) | Time | Yield^{b} |
| 1 | (IPr)CuCl | 5 mol % | water/tBuOH (3) | 18 h | 18% |
| 2 | (IMes)CuCl | 5 mol % | water/tBuOH (3) | 18 h | 65% |
| 3 | (SIMes)CuCl | 5 mol % | water/tBuOH (3) | 18 h | 93% |
| 4 | (SIMes)CuBr | 5 mol % | water/tBuOH (3) | 9 h | 95% |
| 5 | (SIMes)CuBr | 5 mol % | water (1) | 0.5 h | 98% |
| 6 | (SIMes)CuBr | 0.8 mol % | none | 0.3 h | 98% |
| 7 | CuBr | 5 mol % | none | 1 h | 0 |

^{a}Mol % is based on copper.
^{b}Isolated yields are the average of at least two runs.

### Example 2

### General Procedure for the [3+2] Cycloaddition of Azides and Terminal Alkynes—Cu Halides

In a vial fitted with a screw cap, an organic azide (1.0 mmol), an alkyne (1.05 mmol) and [(SIMes)CuBr] (3.6 mg if 0.8 mol % or 9 mg if 2 mol %) were loaded. The reaction was allowed to proceed at room temperature (unless otherwise noted; see Table 2) and monitored by ¹H NMR analysis of aliquots. After total consumption of the starting azide, the solid product was collected by filtration and washed with water and pentane. When the corresponding triazole was an oil or a low-melting point solid, the reaction mixture was poured into an aqueous NH₄Cl/diethyl ether mixture. After extraction of the aqueous phase with diethyl ether, the combined organic layers were washed with brine, dried over magnesium sulfate, filtered and evaporated. In all runs, the crude products were estimated to be greater than 95% pure by ¹H NMR. Results are summarized in Table 2. Reported yields are isolated yields and are the average of at least two runs.

TABLE 2

| Run | Azide | Alkyne | Amt. catalyst^{a} | Temp. | Time | | Yield^{b} |
|---|---|---|---|---|---|---|---|
| a | benzyl azide | phenylacetylene | 0.8 mol % | room | 20 | min. | 98% |
| b | benzyl azide | ethyl propiolate | 0.8 mol % | room | 2 | h | 91% |
| c | benzyl azide | trimethylsilylacetylene | 0.8 mol % | 45° C. | 45 | min. | 98% |
| d | 4-cyanobenzyl azide | phenylacetylene | 0.8 mol % | room | 30 | min. | 93% |
| e | 4-nitrobenzyl azide | phenylacetylene | 0.8 mol % | room | 45 | min. | 89% |
| f | 4-nitrobenzyl azide | 1-ethynylcyclohexene | 0.8 mol % | room | 1.5 | h | 93% |
| g | heptyl azide | phenylacetylene | 0.8 mol % | room | 25 | min. | 93% |
| h | heptyl azide | 4-methoxyphenylacetylene | 0.8 mol % | room | 15 | min. | 93% |
| i | heptyl azide | 3-fluorophenylacetylene | 0.8 mol % | room | 10 | min. | 89% |
| j | heptyl azide | 3,3-dimethyl-1-butyne | 2 mol % | room | 5 | h | 95% |
| k | phenyl azide | phenylacetylene | 0.8 mole % | room | 1.5 | h | 86% |
| l | 2-phenylethyl azide | 2-methyl-3-butyn-2-ol | 0.8 mole % | room | 4 | h | 94% |
| m | 2-[2-(1,3- | phenylacetylene | 0.8 mole % | room | 1 | h | 92% |
| | dioxolanyl)]-ethyl | | | | | | |
| | azide | | | | | | |

^{a}Mol % is based on copper.
^{b}Isolated yields are the average of at least two runs.

Details, including spectroscopic and analytical data, for some of the 1,2,3-triazoles prepared in Example 2 follow.

4-Cyclohexenyl-1-(4-nitrobenzyl)-1H-1,2,3-triazole (Run f). Using the general procedure above, from 0.176 g of 4-(azidomethyl)-4-nitrobenzene and 0.118 mL of 1-ethynylcyclohex-1-ene, 0.263 g of the title compound was isolated as a light yellow solid after filtration (93% yield).

¹H NMR (300 MHz, CDCl₃): δ=8.17 (d, J=8.6 Hz, 2H, H^{Ar}), 7.41 (s, 1H, NCH═), 7.40 (d, J=8.6 Hz, 2H, H^{Ar}), 6.51 (broad s, 1H, ═CHCH₂), 5.62 (s, 2H, NCH₂), 2.40-2.23 (m, 2H, cyclohexenyl), 2.23-2.11 (m, 2H, cyclohexenyl), 1.80-1.56 (m, 4H, cyclohexenyl); ¹³C NMR (75 MHz, CDCl₃): δ=150.2 (C, NC═), 147.8 (C, C^{Ar}), 142.1 (C, C^{Ar}), 128.3 (CH, C^{Ar}), 126.8 (C, C═CHCH₂), 125.5 (CH, C═CHCH₂), 124.1 (CH, C^{Ar}), 118.5 (CH, NCH═), 52.8 (CH₂, NCH₂), 26.2 (CH₂), 25.1 (CH₂), 22.2 (CH₂), 22.0 (CH₂); Elemental analysis calcd for C₁₅H₁₆N₄O₂ (284.31): C, 63.37; H, 5.67; N, 19.71. Found: C, 63.49; H, 5.36; N, 19.35.

1-Heptyl-4-phenyl-1H-1,2,3-triazole (Run g). Using the general procedure above, from 0.176 g of 1-azidoheptane and 0.11 mL of phenylacetylene, 0.225 g of the title compound was isolated as a white solid after filtration (93% yield).

¹H NMR (300 MHz, CDCl₃): δ=7.85 (d, J=7.1 Hz, 2H, H^{Ar}), 7.75 (s, 1H, NCH═), 7.48-7.40 (m, 2H, H^{Ar}), 7.40-7.29 (m, 1H, H^{Ar}), 4.40 (t, J=7.2 Hz, NCH₂), 2.04-1.88 (m, 2H, NCH₂CH₂), 1.43-1.19 (m, 8H, CH₂CH₂CH₂CH₂CH₃), 0.87 (t, J=6.7 Hz, CH₃); ¹³C NMR (75 MHz, CDCl₃): δ=147.5 (C, NC═), 130.6 (C, C^{Ar}), 128.7 (CH, C^{Ar}), 127.9 (CH, C^{Ar}), 125.5 (CH, C^{Ar}), 119.4 (CH, NCH═), 50.2 (CH₂, NCH₂), 31.4 (CH₂, heptyl), 30.2 (CH₂, heptyl), 28.6 (CH₂, heptyl), 26.3 (CH₂, heptyl), 22.4 (CH₂, heptyl), 13.9 (CH₃); Elemental analysis calcd for C₁₅H₂₁N₃ (243.35): C, 74.03; H, 8.70; N, 17.27. Found: C, 73.79; H, 8.60; N, 17.18.

1-Heptyl-4-(4-methoxyphenyl)-1H-1,2,3-triazole (Run h). Using the general procedure above, from 0.176 g of 1-azidoheptane and 0.136 mL of 1-ethynyl-4-methoxybenzene, 0.255 g of the title compound was isolated as a white solid after filtration (93% yield).

¹H NMR (300 MHz, CDCl₃): δ=7.76 (d, J=8.8 Hz, 2H, H^{Ar}), 7.66 (s, 1H, NCH═), 6.96 (d, J=8.8 Hz, 2H, H^{Ar}), 4.37 (t, J=7.2 Hz, NCH₂), 3.84 (s, 3H, OCH₃), 2.00-1.83 (m, 2H, NCH₂CH₂), 1.42-1.21 (m, 8H, CH₂CH₂CH₂CH₂CH₃), 0.88 (t, J=6.8 Hz, CH₃); ¹³C NMR (75 MHz, CDCl₃): δ=159.4 (C, C^{Ar}), 147.5 (C, NC═), 126.9 (CH, C^{Ar}), 123.4 (C, C^{Ar}), 118.6 (CH, NCH═), 114.1 (CH, C^{Ar}), 55.2 (CH₂, NCH₂), 50.3 (CH₃, OCH₃), 31.5 (CH₂, heptyl), 30.3 (CH₂, heptyl), 28.6 (CH₂, heptyl), 26.4 (CH₂, heptyl), 22.5 (CH₂, heptyl), 14.0 (CH₂CH₃); Elemental analysis calcd for C₁₆H₂₃N₃O (273.37): C, 70.30; H, 8.48; N, 15.37. Found: C, 69.98; H, 8.79; N, 15.24.

4-(3-Fluorophenyl)-1-heptyl-1H-1,2,3-triazole (Run i). Using the general procedure above, from 0.176 g of 1-azidoheptane and 0.121 mL of 1-ethynyl-3-fluorobenzene, 0.233 g of the title compound was isolated as an off-white solid after extraction (89% yield).

¹H NMR (300 MHz, CDCl₃): δ=7.76 (s, 1H, NCH═), 7.65-7.52 (m, 2H, H^{Ar}), 7.43-7.34 (m, 1H, H^{Ar}), 7.08-6.97 (m, 1H, H^{Ar}), 4.41 (t, J=7.3 Hz, NCH₂), 2.02-1.89 (m, 2H, NCH₂CH₂), 1.43-1.20 (m, 8H, CH₂CH₂CH₂CH₂CH₃), 0.89 (t, J=6.7 Hz, CH₃); ¹³C NMR (75 MHz, CDCl₃): δ=160.4 (d, J=244 Hz, C, C—F), 146.5 (C, NC═), 132.8 (d, J=8.5 Hz, C, C^{Ar}), 130.3 (d, J=8.5 Hz, CH, C^{Ar}), 121.2 (CH, C^{Ar}), 119.8 (CH, NCH═), 114.7 (d, J=21 Hz, CH, C^{Ar}), 112.4 (d, J=23 Hz, CH, C^{Ar}), 50.4 (CH₂, NCH₂), 31.4 (CH₂, heptyl), 30.2 (CH₂, heptyl), 28.6 (CH₂, heptyl), 26.3 (CH₂, heptyl), 22.4 (CH₂, heptyl), 13.9 (CH₃); Elemental analysis calcd for C₁₅H₂₀FN₃ (261.34): C, 68.94; H, 7.71; N, 16.08. Found: C, 68.87; H, 7.99; N, 15.85.

4-tert-Butyl-1-heptyl-1H-1,2,3-triazole (Run j). Using the general procedure above, from 0.176 g of 1-azidoheptane and 0.13 mL of 3,3-dimethylbut-1-yne and 2 mol % of [(SIMes)CuBr], 0.212 g of the title compound was isolated as a light yellow oil after extraction (95% yield).

¹H NMR (300 MHz, CDCl₃): δ=7.19 (s, 1H, NCH═), 4.21 (t, J=7.4 Hz, 2H, NCH₂), 1.84-1.74 (m, 2H, NCH₂CH₂), 1.33-1.21 (m, CH₂CH₂CH₂CH₂CH₃) and 1.26 (s, CCH₃) (17H), 0.80 (t, J=6.8 Hz, CH₃); ¹³C NMR (75 MHz, CDCl₃): δ=157.2 (C, NC═), 118.1 (CH, NCH═), 49.8 (CH₂, NCH₂), 31.3 (CH₂, heptyl), 30.4 (C, CCH₃), 30.1 (CH₃, CCH₃), 28.4 (CH₂, heptyl), 26.2 (CH₂, heptyl), 22.2 (CH₂, heptyl), 13.8 (CH₂CH₃); Elemental analysis calcd for C₁₃H₂₅N₃ (223.36): C, 69.91; H, 11.28; N, 18.81. Found: C, 70.01; H, 11.56; N, 18.76.

2-(1-Phenethyl-1H-1,2,3-triazol-4-yl)propan-2-ol (Run l). Using the general procedure above, from 0.147 g of (2-azidoethyl)benzene and 0.11 mL of 2-methylbut-3-yn-2-ol, 0.216 g of the title compound was isolated as a white solid after filtration (94% yield).

¹H NMR (300 MHz, CDCl₃): δ=7.39-7.21 (m, 3H, H^{Ar}), 7.19 (s, 1H, NCH═), 7.18-7.02 (m, 2H, H^{Ar}), 4.54 (t, J=7.6 Hz, PhCH₂), 3.19 (t, J=7.6 Hz, NCH₂), 2.99 (s broad, 1H, OH), 1.59 (s, 6H, CH₃); ¹³C NMR (75 MHz, CDCl₃): δ=155.3 (C, NC═), 137.0 (C, C^{Ar}), 128.7 (CH, C^{Ar}), 128.6 (CH, C^{Ar}), 127.0 (CH, C^{Ar}), 119.5 (CH, NCH═), 68.3 (C, COH), 51.5 (CH₂, NCH₂), 36.7 (CH₂, PhCH₂), 30.4 (CH₃); Elemental analysis calcd for C₁₃H₁₇N₃O (231.29): C, 67.51; H, 7.41; N, 18.17. Found: C, 67.45; H, 7.48; N, 17.87.

1-[2-(1,3-Dioxolan-2-yl)ethyl]-4-phenyl-1H-1,2,3-triazole (Run m). Using the general procedure above, from 0.143 g of (2-azidoethyl)-1,3-dioxolane and 0.11 mL of phenylacetylene, 0.226 g of the title compound was isolated as a white solid after filtration (92% yield).

¹H NMR (300 MHz, CDCl₃): δ=7.87-7.73 (m, 3H, H^{Ar}+NCH═), 7.47-7.28 (m, 3H, H^{Ar}), 4.94 (t, J=4.3 Hz, 1H, OCHO), 4.55 (t, J=7.2 Hz, 2H, NCH₂), 4.04-3.92 (m, 2H, OCH₂), 3.92-3.84 (m, 2H, OCH₂), 2.37-2.27 (m, 2H, NCH₂CH₂); ¹³C NMR (75 MHz, CDCl₃): δ=147.4 (C, NC═), 130.6 (C, C^{Ar}), 128.7 (CH, C^{Ar}), 128.0 (CH, C^{Ar}), 125.6 (CH, C^{Ar}), 119.8 (CH, NCH═), 101.4 (CH, OCHO), 65.0 (CH₂, CH₂O), 45.3 (CH₂, NCH₂), 34.0 (CH₂, NCH₂CH₂); Elemental analysis calcd for C₁₃H₁₅N₃O₂ (245.28): C, 63.66; H, 6.16; N, 17.13. Found: C, 63.82; H, 6.22; N, 16.86.

### Example 3

### General Procedure for the [3+2] Cycloaddition of In Situ-Generated Azides and Terminal Alkynes—Cu Halides

The procedure described above for Example 2 was followed using an alkyl halide (1.0 mmol), NaN₃ (68 mg, 1.05 mmol), and an alkyne (1.05 mmol) in water (1 mL). For all runs, 5 mol % of [(SIMes)CuBr] was used. Results are summarized in Table 3.

TABLE 3

| Run | Alkyl halide | Alkyne | Temp. | Time | Yield^{a} |
|---|---|---|---|---|---|
| 1 | benzyl bromide | phenylacetylene | room | 1 h | 92% |
| 2 | benzyl chloride | phenylacetylene | room | 5 h | 93% |
| 3 | benzyl chloride | phenylacetylene | 70° C. | 0.3 h | 94% |
| 4 | 4-cyanobenzyl | phenylacetylene | room | 2 h | 97% |
| | bromide | | | | |
| 5 | 4-nitrobenzyl | phenylacetylene | room | 1.5 h | 98% |
| | bromide | | | | |
| 6 | heptyl bromide | 3-fluoro- | 45° C. | 0.5 h | 92% |
| | | phenylacetylene | | | |
| 7 | methyl iodide | phenylacetylene | room | 2 h | 90% |

^{a}Isolated yields are the average of at least two runs.

Details for one of the 1,2,3-triazoles prepared in Example 3 follow.

4-(3-Fluorophenyl)-1-heptyl-1H-1,2,3-triazole (Run 6). Using the general procedure above, from 0.157 mL of 1-bromoheptane and 0.121 mL of 1-ethynyl-3-fluorobenzene, 0.240 g of the title compound was isolated as a white solid after extraction (92% yield). Spectroscopic and analytical data are as reported in Run i of Example 2.

### Example 4

### General Procedure for the [3+2] Cycloaddition of Azides and Internal Alkynes—Cu Halides

In a vial fitted with a screw cap, azide (1.0 mmol), 3-hexyne (0.120 mL, 1.05 mmol) and [(SIMes)CuBr] (22 mg, 5 mol %) were loaded. The reaction was allowed to proceed at 70° C. for 48 h. The reaction mixture was allowed to cool down and poured on an aqueous NH₄Cl/diethyl ether mixture. After extraction of the aqueous phase with diethyl ether, the combined organic layers were washed with brine, dried over magnesium sulfate, filtered and evaporated. Due to their low melting point, the product triazoles could not be purified by recrystallization, and the crude products were purified by flash chromatography on silica gel. Results are summarized in Table 4.

TABLE 4

| Run | Azide | Alkyne | Amt. catalyst | Yield^{a} |
|---|---|---|---|---|
| o-1 | benzyl azide | 3-hexyne | none | <5% |
| o-2 | benzyl azide | 3-hexyne | 5 mol % | 80% |
| p-1 | 4-nitrobenzyl azide | 3-hexyne | none | <5% |
| p-2 | 4-nitrobenzyl azide | 3-hexyne | 5 mol % | 59% |

^{a}Yield is from NMR conversion.

Details, including spectroscopic and analytical data, for some of the 1,2,3-triazoles prepared in Example 4 follow.

1-Benzyl-4,5-diethyl-1H-1,2,3-triazole (Run o-2). Using the general procedure above, from 0.133 g of benzyl azide and after purification by flash chromatography on silica gel (pentane/diethyl ether: 1:1), 0.153 g of the title compound was isolated as a colorless oil (71% yield). ¹H NMR (300 MHz, CDCl₃): δ=7.42-7.27 (m, 3H, H^{Ar}), 7.21-7.18 (m, 2H, H^{Ar}), 5.48 (s, 2H, NCH₂), 2.65 (q, J=7.6 Hz, CH₂CH₃), 2.52 (q, J=7.6 Hz, CH₂CH₃), 1.28 (t, J=7.6 Hz, CH₂CH₃), 0.96 (t, J=7.6 Hz, CH₂CH₃); ¹³C NMR (75 MHz, CDCl₃): δ=146.39 (C, NC═), 146.34 (C, NC═), 135.4 (C, C^{Ar}), 128.88 (CH, C^{Ar}), 128.81 (CH, C^{Ar}), 128.1 (CH, C^{Ar}), 51.8 (CH₂, NCH₂), 18.5 (CH₂, CH₂CH₃), 15.9 (CH₂, CH₂CH₃), 14.2 (CH₃), 13.3 (CH₃); Elemental analysis calcd for C₁₃H₁₇N₃ (215.29): C, 72.52; H, 7.96; N, 19.52. Found: C, 72.43; H, 7.83; N, 19.45.

4,5-Diethyl-1-(4-nitrobenzyl)-1H-1,2,3-triazole (Run p-2). Using the general procedure above, from 0.176 g of 4-(azidomethyl)-4-nitrobenzene and after purification by flash chromatography on silica gel (diethyl ether), 0.130 g of the title compound was isolated as a yellow oil (48% yield).

¹H NMR (300 MHz, CDCl₃): δ=8.21 (d, J=8.7 Hz, H^{Ar}), 7.30 (d, J=8.7 Hz, H^{Ar}), 5.58 (s, 2H, NCH₂), 2.67 (q, J=7.5 Hz, CH₂CH₃), 2.55 (q, J=7.5 Hz, CH₂CH₃), 1.30 (t, J=7.5 Hz, CH₂CH₃), 1.01 (t, J=7.5 Hz, CH₂CH₃); ¹³C NMR (75 MHz, CDCl₃): δ=147.71 (C), 147.66 (C), 147.63 (C), 142.6 (C, C^{Ar}), 127.7 (CH, C^{Ar}), 124.1 (CH, C^{Ar}), 50.7 (CH₂, NCH₂), 18.4 (CH₂, CH₂CH₃), 15.8 (CH₂, CH₂CH₃), 14.1 (CH₃), 13.5 (CH₃); Elemental analysis calcd for C₁₃H₁₆N₄O₂ (260.29): C, 59.99; H, 6.20; N, 21.52. Found: C, 60.34; H, 6.33; N, 21.76.

### Example 5

Several runs were performed using benzyl azide and phenylacetylene, using different copper tetrafluoroborate or hexafluorophosphate catalysts. In a vial fitted with a screw cap, benzyl azide (1.0 mmol), phenylacetylene (1.05 mmol), water, and the catalyst (2 mol %, based on copper) were loaded. The reaction was allowed to proceed at room temperature and monitored by ¹H NMR analysis of aliquots. No general trend for the reactivity of the complexes was found. Results are summarized in Table 5.

TABLE 5

| Run | Catalyst | Time | Conversion^{a} | Run | Catalyst | Time | Conversion^{a} |
|---|---|---|---|---|---|---|---|
| 1a | [(IPr)₂Cu]PF₆ | 18 h | 71% | 1b | [(IPr)₂Cu]BF₄ | 8 h | 100% |
| 2a | [(SIPr)₂Cu]PF₆ | 5 h | 100% | 2b | [(SIPr)₂Cu]BF₄ | 5 h | 100% |
| 3a | [(IMes)₂Cu]PF₆ | 6 h | 100% | 3b | [(IMes)₂Cu]BF₄ | 6 h | 100% |
| 4a | [(SIMes)₂Cu]PF₆ | 18 h | 5% | 4b | [(SIMes)₂Cu]BF₄ | 18 h | 13% |
| 5a | [(ICx)₂Cu]PF₆ | 1.5 h | 99% | 5b | [(ICx)₂Cu]BF₄ | 5 h | 95% |
| 6a | [(IAd)₂Cu]PF₆ | 5 h | 100% | 6b | [(IAd)₂Cu]BF₄ | 3 h | 100% |
| 7a | [(ItBu)₂Cu]PF₆ | 18 h | 76% | 7b | [(ItBu)₂Cu]BF₄ | 18 h | 35% |

^{a}Conversions are the average of at least two runs.

### Example 6

### General Procedure for the [3+2] Cycloaddition of Azides and Terminal Alkynes—Cu BF₄/PF₆

In a vial fitted with a screw cap, an organic azide (1.0 mmol), an alkyne (1.05 mmol) and [(ICx)₂Cu]PF₆ (0.5 mol %) were loaded. The reaction was allowed to proceed at room temperature and monitored by ¹H NMR analysis of aliquots. After total consumption of the starting azide, solid products were collected by filtration or evaporation. Results are summarized in Table 6. Reported yields are isolated yields.

TABLE 6

| Run | Azide | Alkyne | Time | | Yield^{a} |
|---|---|---|---|---|---|
| a | benzyl azide | phenylacetylene | 5 | min. | 99% |
| b | benzyl azide | n-hexyne | 45 | min. | 93% |
| c | benzyl azide | 2-ethynylpyridine | 5 | min. | 97% |
| d | 4-cyanobenzyl azide | 3-ethynylpyridine | 5 | min | 97% |
| e | 4-nitrobenzyl azide | 3-methyl-3-hydroxy-1-butyne | 4 | h | 91% |
| f | heptyl azide | phenylacetylene | 5 | min. | 99% |
| g | heptyl azide | 4-methoxyphenylacetylene | 9 | h | 95% |
| h | phenyl azide | phenylacetylene | 5 | min. | 99% |
| i | phenyl azide | 5-chloropentyne | 25 | min. | 98% |
| j | 2-phenylethyl azide | 3-(dimethylamino)propyne | 5 | min. | 99% |
| k | 4-nitrobenzyl azide | but-3-yn-2-one | 5 | h | 96% |
| l | 4-nitrobenzyl azide | 1-ethynylcyclohexene | 10 | min. | 92% |
| m | 4-cyanobenzyl azide | prop-2-yn-1-ol | 5 | h | 92% |
| n | 2-[2-(1,3-dioxolanyl)]-ethyl azide | ethyl propiolate | 7 | h | 96% |
| o | 3-cyanopropyl azide | ethyl 2-propynoate | 7 | h | 96% |

^{a}Yields are the average of at least two runs.

### Example 7

In a vial fitted with a screw cap, an organic azide (1.0 mmol), an alkyne (1.05 mmol) and [(ICx)₂Cu]PF₆ were loaded. Reactions were performed at different catalyst loadings and at different temperatures; see Table 7. The reaction was allowed to proceed at the selected temperature and monitored by ¹H NMR analysis of aliquots. After total consumption of the starting azide, solid products were collected by filtration or evaporation. Results are summarized in Table 7. Reported conversions were determined by ¹H NMR.

TABLE 7

| | | | Amt. | | | | |
|---|---|---|---|---|---|---|---|
| Run | Azide | Alkyne | catalyst^{a} | Temp. | Time | Conversion^{b} | TON^{c} |
| a-1 | benzyl azide | phenylacetylene | 50 ppm | room | 48 h | 80% | 16000 |
| a-2 | | | 50 ppm | 40° C. | 8 h | 89% | 17800 |
| a-3 | | | 40 ppm | 50° C. | 4 h | 81% | 20250 |
| d-1 | benzyl azide | 3-ethynylpyridine | 75 ppm | room | 6 h | 91% | 12133 |
| f-1 | heptyl azide | phenylacetylene | 200 ppm | room | 20 h | 72% | 3600 |
| i-1 | phenyl azide | 5-chloropentyne | 300 ppm | room | 43 h | 85% | 2833 |
| i-2 | | | 100 ppm | 40° C. | 18 h | 70% | 7000 |
| j-1 | 2-phenylethyl azide | 3-(dimethylamino)propyne | 300 ppm | room | 40 h | 45% | 1500 |
| j-2 | | | 100 ppm | 40° C. | 18 h | 71% | 7100 |

^{a}Amount is based on copper.
^{b}Conversions are the average of at least two runs.
^{c}TON is turn over number.

The invention may comprise, consist, or consist essentially of the materials and/or procedures recited herein.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about”, the claims include equivalents to the quantities.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition.

Each and every patent, patent application, and printed publication referred to above is incorporated herein by reference in toto to the fullest extent permitted as a matter of law.

This invention is susceptible to considerable variation in its practice. Therefore, the foregoing description is not intended to limit, and should not be construed as limiting, the invention to the particular exemplifications presented hereinabove.

That which is claimed is:

1. A process which comprises contacting, in a reaction zone, at least one organic azide, at least one alkyne, and at least one N-heterocyclic carbene copper compound in which the ligands are either (i) a halide and an N-heterocyclic carbene or (ii) two N-heterocyclic carbenes and a BF₄⁻ or PF₆⁻ anion, to form a 1,2,3-triazole in which at least the 1 and 4 positions each has a substituent, wherein said N-heterocyclic carbene is either an imidazol-2-ylidene in which the 1 and the 3 positions each has a substituent which has at least one carbon atom, or a 4,5-dihydro-imidazol-2-ylidene in which the 1 and the 3 positions each has a substituent which has at least one carbon atom.

2. A process as in claim 1 wherein said organic azide is an aryl azide or an aralkyl azide.

3. A process as in claim 1 wherein said organic azide is phenyl azide, 4-cyanophenyl azide, 4-nitrophenyl azide, benzyl azide, 2-phenylethyl azide, 4-cyanobenzyl azide, 4-nitrobenzyl azide, methyl azide, 3-cyanopropyl azide, heptyl azide, 3,3-diethoxypropyl azide, or 2-[2-(1,3-dioxolanyl)]-ethyl azide.

4. A process as in claim 1 wherein said alkyne has at least one carbon-carbon double bond, ether group, ketyl group, ester group, hydroxyl group, chlorine atom, fluorine atom, nitrogen atom, or trihydrocarbylsilyl group.

5. A process as in claim 1 wherein said alkyne is a terminal alkyne.

6. A process as in claim 5 wherein said terminal alkyne is selected from the group consisting of 3,3-dimethyl-1-butyne, 1-ethynylcyclohexene, phenylacetylene, 5-chloropentyne, 2-methyl-3-butyn-2-ol, 2-propyn-1-ol, 4-methoxyphenylacetylene, ethyl propiolate, (trimethylsilyl)acetylene, 3-(dimethylamino)propyne, 2-ethynylpyridine, and 3-ethynylpyridine.

7. A process as in claim 1 wherein said alkyne is an internal alkyne.

8. A process as in claim 7 wherein said internal alkyne is 3-hexyne or diphenylacetylene.

9. A process as in claim 1 wherein said N-heterocyclic carbene copper compound is a N-heterocyclic carbene copper halide which is a N-heterocyclic carbene copper chloride or a N-heterocyclic carbene copper bromide.

10. A process as in claim 1 wherein said N-heterocyclic carbene copper compound has two N-heterocyclic carbenes and a BF₄⁻ or PF₆⁻ anion.

11. A process as in claim 1 wherein said N-heterocyclic carbene is a 4,5-dihydro-imidazol-2-ylidene in which the 1 and the 3 positions each has a substituent which has at least one carbon atom.

12. A process as in claim 1 wherein each substituent, independently, on the N-heterocyclic carbene is an aryl group or an alkyl group having at least 3 carbon atoms.

13. A process as in claim 12 wherein when said substituents on the N-heterocyclic carbene are alkyl groups, each alkyl group is a secondary or tertiary group, and when said substituents on the N-heterocyclic carbene are aryl groups, each aryl group is substituted by an alkyl group in each ortho position.

14. A process as in claim 1 wherein said N-heterocyclic carbene is N,N′-bis[2,6-di(isopropyl)phenyl]-imidazol-2-ylidene, N,N′-bis(2,4,6-trimethylphenyl)-imidazol-2-ylidene, N,N′-bis[2,6-di(isopropyl)phenyl]-4,5-dihydro-imidazol-2-ylidene, N,N′-bis(2,4,6-trimethylphenyl)-4,5-dihydro-imidazol-2-ylidene, N,N′-di(cyclohexyl)-imidazol-2-ylidene, or N,N′-di(adamantyl)-imidazol-2-ylidene.

15. A process as in claim 1 wherein said N-heterocyclic carbene copper compound is a N-heterocyclic carbene copper halide which is [N,N′-bis{2,6-di(isopropyl)phenyl}-imidazol-2-ylidene]copper chloride, [N,N′-bis{2,6-di(isopropyl)phenyl}-imidazol-2-ylidene]copper bromide, [N,N′-bis(2,4,6-trimethylphenyl)-imidazol-2-ylidene]copper chloride, [N,N′-bis(2,4,6-trimethylphenyl)-imidazol-2-ylidene]copper bromide, [N,N′-bis(2,4,6-trimethylphenyl)-4,5-dihydro-imidazol-2-ylidene]copper chloride, or [N,N′-bis(2,4,6-trimethylphenyl)-4,5-dihydro-imidazol-2-ylidene]copper bromide.

16. A process as in claim 1 wherein said N-heterocyclic carbene copper compound has two N-heterocyclic carbenes and a BF₄⁻ or PF₆⁻ anion, and which compound is bis(N,N′-di(cyclohexyl)-imidazol-2-ylidene)copper hexafluorophosphate, bis(N,N′-di(cyclohexyl)-imidazol-2-ylidene)copper tetrafluoroborate, bis(N,N′-di(adamantyl)-imidazol-2-ylidene) copper hexafluorophosphate, bis(N,N′-di(adamantyl)-imidazol-2-ylidene) copper tetrafluoroborate, bis(N,N′-bis[2,6-di(isopropyl)phenyl]-imidazol-2-ylidene)copper hexafluorophosphate, bis(N,N′-bis[2,6-di(isopropyl)phenyl]-imidazol-2-ylidene)copper tetrafluoroborate, bis(N,N′-bis(2,4,6-trimethylphenyl)-imidazol-2-ylidene)copper hexafluorophosphate, bis(N,N′-bis(2,4,6-trimethylphenyl)-imidazol-2-ylidene)copper tetrafluoroborate, bis(N,N′-bis[2,6-di(isopropyl)phenyl]-4,5-dihydro-imidazol-2-ylidene)copper hexafluorophosphate, bis(N,N′-bis[2,6-di(isopropyl)phenyl]-4,5-dihydro-imidazol-2-ylidene)copper tetrafluoroborate, bis(N,N′-bis(2,4,6-trimethylphenyl)-4,5-dihydro-imidazol-2-ylidene)copper hexafluorophosphate, or bis(N,N′-bis(2,4,6-trimethylphenyl)-4,5-dihydro-imidazol-2-ylidene)copper tetrafluoroborate.

17. A process as in claim 1 wherein water is present in said reaction zone during said process.

18. A process as in claim 5 wherein water is present in said reaction zone during said process.

19. A process as in claim 1 wherein either said organic azide is formed in a reaction zone in which the alkyne and the catalyst are already present, or said organic azide is formed in a reaction zone to which the alkyne and the catalyst are being or will be fed.

20. A process as in claim 19 wherein said organic azide is formed from an organic halide and an alkali metal azide.

21. A process as in claim 1 wherein said organic azide is an aryl azide or an aralkyl azide; wherein said alkyne is a terminal alkyne; and wherein said N-heterocyclic carbene copper compound is a N-heterocyclic carbene copper halide which is [N,N′-bis{2,6-di(isopropyl)phenyl}-imidazol-2-ylidene]copper chloride, [N,N′-bis{2,6-di(isopropyl)phenyl}-imidazol-2-ylidene]copper bromide, [N,N′-bis(2,4,6-trimethylphenyl)-imidazol-2-ylidene]copper chloride, or [N,N′-bis(2,4,6-trimethylphenyl)-imidazol-2-ylidene]copper bromide.

22. A process as in claim 21 wherein water is present in said reaction zone during said process.

23. A process as in claim 16 wherein said organic azide is an aryl azide or an aralkyl azide; and wherein said alkyne is a terminal alkyne.

24. A process as in claim 1 wherein said organic azide is an aryl azide or an aralkyl azide; wherein said alkyne is an internal alkyne; and wherein said N-heterocyclic carbene copper compound is a N-heterocyclic carbene copper halide which is [N,N′-bis{2,6-di(isopropyl)phenyl}-imidazol-2-ylidene]copper chloride, [N,N′-bis{2,6-di(isopropyl)phenyl}-imidazol-2-ylidene]copper bromide, [N,N′-bis(2,4,6-trimethylphenyl)-imidazol-2-ylidene]copper chloride, or [N,N′-bis(2,4,6-trimethylphenyl)-imidazol-2-ylidene]copper bromide.

25. A process as in claim 16 wherein said organic azide is an aryl azide or an aralkyl azide; and wherein said alkyne is an internal alkyne.
